(19) United States
(12) Patent Application Publication
(10) Pub. No.: US 2018/0228994 A1
(43) Pub. Date: Aug. 16, 2018

(54) EXTRUDED PORTABLE OXYGEN CONCENTRATOR ASSEMBLY

(71) Applicant: Caire Inc., Ball Ground, GA (US)

(72) Inventor: Beau Hart, Ball Ground, GA (US)

(21) Appl. No.: 15/888,452

(22) Filed: Feb. 5, 2018

Related U.S. Application Data
(60) Provisional application No. 62/458,955, filed on Feb. 14, 2017.

(51) Int. Cl. A61M 16/10

(52) U.S. Cl. CPC A61M 16/101; A61M 16/105; A61M 2205/75; A61M 2205/42; A61M 2202/0208

(57) ABSTRACT

An oxygen concentrator includes at least one extruded structural component. The extruded structural component is both a mounting for other components of the oxygen concentrator and at least part of an external surface of the oxygen concentrator.

### CROSS REFERENCE TO RELATED APPLICATIONS

The present application claims priority to U.S. Provisional Patent Application Ser. No. 62/458,955, filed Feb. 14, 2017, entitled “Extruded Portable Oxygen Concentrator Assembly” the full disclosure of which is incorporated herein by reference.

### BACKGROUND

Disclosed is a portable oxygen concentrator having a structure that includes an extruded frame or housing.

Traditional oxygen concentrators (such as stationary, transportable and/or portable oxygen concentrators) are formed of multiple internal elements such as, for example, compressors, valves, sieve beds, tanks, a controller, etc. The internal components are all housed in an external shell or housing that is typically made of molded plastic or other preformed material. Some models use sheet metal for industrial applications.

As the art and technology for providing therapeutic oxygen has matured and changed, the benefits of mobility for oxygen concentrators has driven more patients to use portable oxygen concentrators (sometimes referred to as POCs). POCs must also include batteries for energy to operate, and must still be as light as possible to make them practical to carry on the patient's body (typically by a shoulder strap). Other design challenges with POCs include heat dissipation and vibration isolation. Management of cost is also a major barrier to adoption of the superior POC for many patients, who must remain home and tethered to a stationary device that is powered from household electricity.

### SUMMARY

Disclosed herein is an improved oxygen concentrator system that is portable and that includes an outer housing that is lightweight and can be efficiently manufactured. The disclosed system eliminates or combines multiple structural components with respect to prior oxygen concentrator systems. An improved system provides reduced cost and weight, as well as improved thermal management and ease of repair relative to prior systems. Together, these improved systems can bring the benefits of POC usage to a greater number of patients.

In one aspect, there is disclosed an oxygen concentrator, comprising at least one extruded structural component, wherein the at least one extruded structural component is both (a) a mounting for other components of the oxygen concentrator and (b) at least part of an external surface of the oxygen concentrator.

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE DRAWINGS

The details of one or more variations of the subject matter described herein are set forth in the accompanying drawings and the description below. Other features and advantages of the subject matter described herein will be apparent from the description and drawings, and from the claims.

FIG. 1A shows a perspective view of an example extruded outer housing of a portable oxygen concentrator (POC).

FIG. 1B shows a top view of the outer housing of FIG. 1.

FIG. 2 shows an exploded view of a POC with various components of the POC arranged next to the extruded outer housing.

FIG. 3 shows the assembled POC of FIG. 2 in a partially transparent state.

FIG. 4 shows a top view of another example of a POC.

FIG. 5 shows a perspective view of another example of a POC.

### DETAILED DESCRIPTION

Before the present subject matter is further described, it is to be understood that this subject matter described herein is not limited to particular embodiments described, as such may of course vary. It is also to be understood that the terminology used herein is for the purpose of describing particular embodiments only, and is not intended to be limiting. Unless defined otherwise, all technical terms used herein have the same meaning as commonly understood by one skilled in the art to which this subject matter belongs.

Disclosed herein is a portable concentrator, such as a portable oxygen concentrator that at least partially includes an outer housing structure that is formed of an extruded material. That is, the housing structure is formed pursuant to an extrusion process wherein a material is pushed through a die of a desired cross-sectional shape to form the structure. The extruded structure can use a complex extrusion, such as of an aluminum material. The extruded structure defines an exterior profile of the POC and defines one or more internal cavities that contain one or more components, such as a molecular sieve (adsorbent) in bed tubes. The extruded structure also provides attachment structures or other engaging means for electronics boards and/or a battery. It may also enclose and isolate a compressor and valves of the POC, along with muffler volumes for mitigating valve and flow noises.

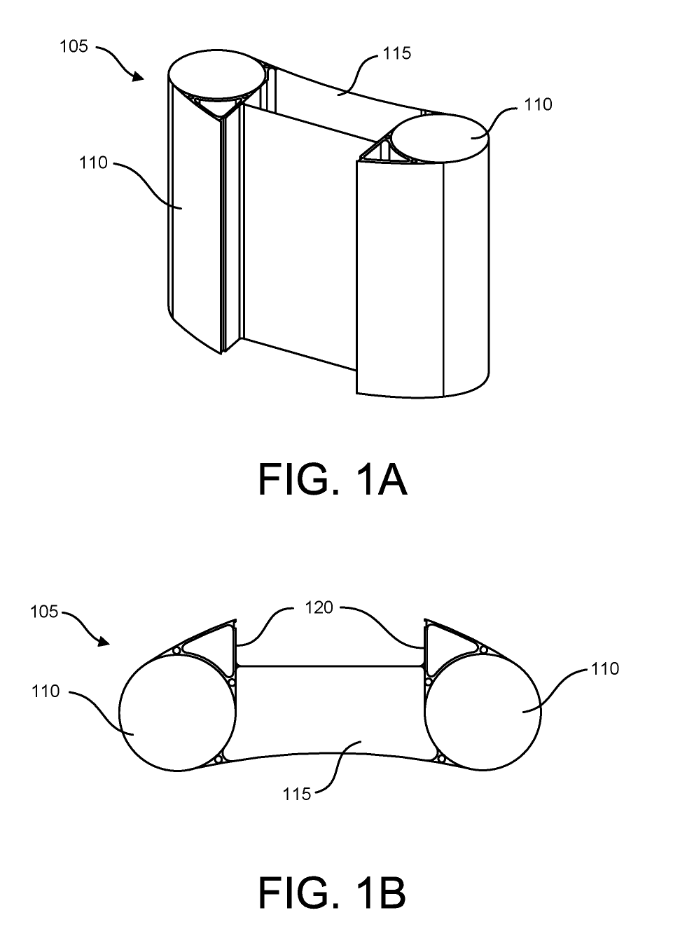

FIG. 1A shows a perspective view of an example outer housing 105 for use with a POC. FIGS. 1A and 1B show an example shape although it should be appreciated that the shape can vary. The outer housing 105 is formed of an extruded material, such as for example aluminum. Pursuant to a manufacturing process, the outer housing is formed or manufactured by pushing the material through a die of desired cross-sectional shape.

FIG. 1B shows a top view of the outer housing 105. The outer housing 105 includes a pair of adsorbent beds sections 110 that are positioned on peripheral ends (relative to the view of FIG. 1B) of the outer housing 105. In the illustrated embodiment, the adsorbent bed sections 110 each form circular shaped zones in the extrusion shape in cross-section when viewed from the top as shown in FIG. 1B. The adsorbent bed sections 110 can have a circular shape in order to bear pressure of the operating cycle of the POC. It should be appreciated that the shape can vary however.

Reference still to FIG. 1A and FIG. 1B, a central section 115 of the POC is positioned between the two adsorbent bed sections 110. In the example embodiment, the central section 115 has a generally rectangular shape (in cross-section when viewed from the top) that defines an internal cavity that may be particularly suited for enclosing one or more components, such as a compressor and valves of the POC. The compressor and valves are positioned within the internal cavity in a finished, assembled device. A pair of generally triangular shaped cavities or compartments 120 are positioned adjacent to the adsorbent beds sections 110. The compartments 120 can serve as housings for components such as mufflers and/or filters of the POC. The housing 105 can also include other sections, such as an open trough that can be can be a receiver for electronics boards and/or a battery.

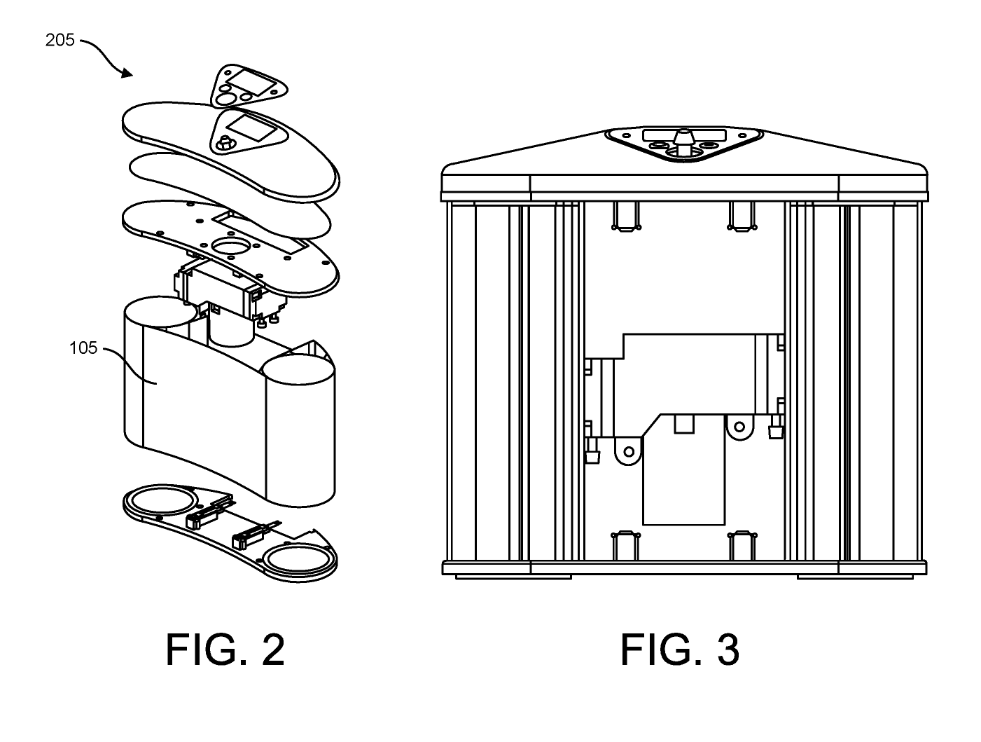

FIG. 2 shows an exploded view of a POC 205 with various components of the POC arranged next to the extruded outer housing 105. The components can be positioned at least partially within the housing 105 in the assembled device. FIG. 3 shows the assembled POC 205 in a partially transparent state. The POC 205 includes a variety of components that are positioned inside the outer housing or that can be positioned next to or on the outer housing. For example, the POC 205 can include a bottom manifold and a battery as well as a compressor, an upper manifold, and electronic components. An upper enclosure can include a user interface with one or more actuators, such as buttons, and can be positioned on a topmost region of the POC immediately above the outer housing 105.

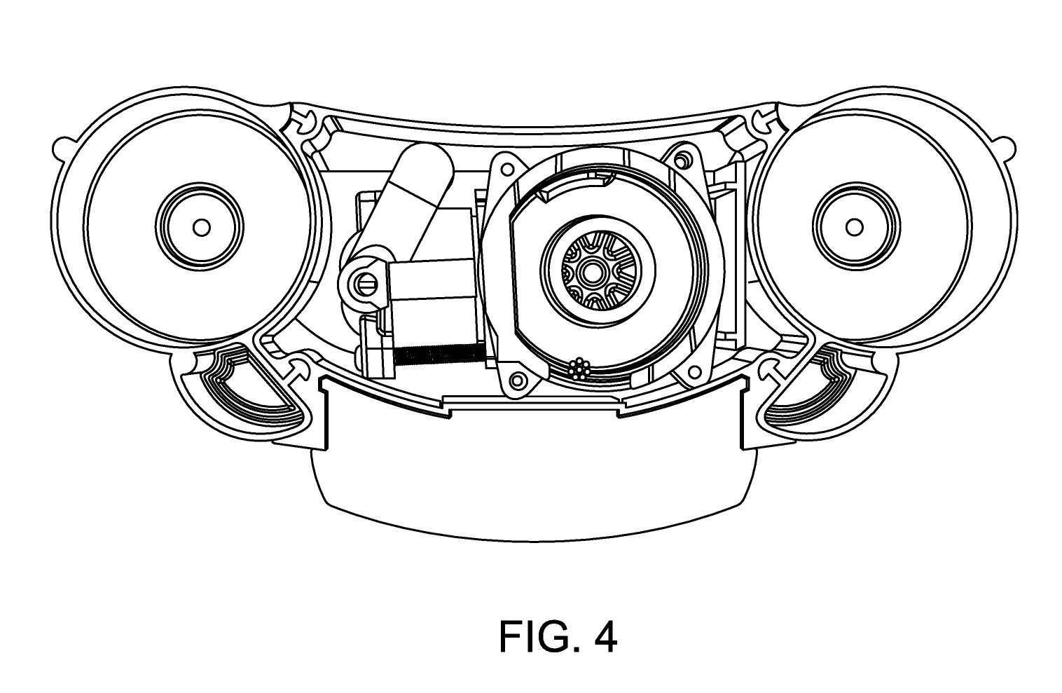

It should be appreciated that variations on the POC assembly are possible without departing from the scope of this disclosure. FIG. 4 shows a version of the POC wherein the outer housing includes three interlocked extrusion assemblies. The interlocked extrusion assemblies comprise structures that engage one another, such as in a sliding manner, such that at least a portion of the outer housing can be removed and also interlocked with another portion of the outer housing. FIG. 4 also shows a compressor positioned inside the outer housing and a side mount battery positioned adjacent the outer housing.

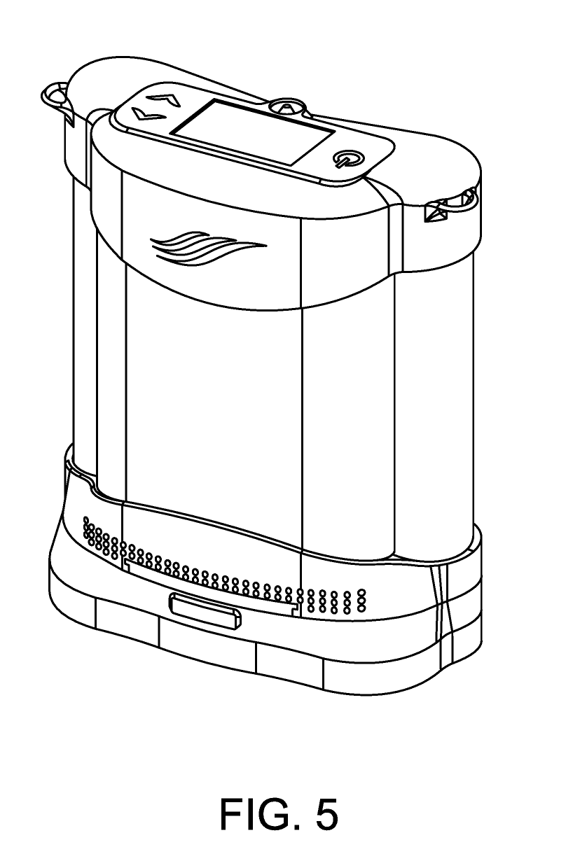

FIG. 5 shows another example wherein a bottom-mount battery is included with the POC. As shown in FIG. 5, the outer housing can be equipped with showing top and bottom caps to finish or otherwise complete the outer surfaces of the extruded structure.

While this specification contains many specifics, these should not be construed as limitations on the scope of an invention that is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

1. An oxygen concentrator, comprising
at least one extruded structural component, wherein the at least one extruded structural component is both (a) a mounting for other components of the oxygen concentrator and (b) at least part of an external surface of the oxygen concentrator.

2. An oxygen concentrator as in claim 1, wherein the extruded structural component forms at least part of a containment for pressure-swing adsorbent beds.

3. An oxygen concentrator as in claim 1, further comprising and the caps that engage with the extruded component.

4. An oxygen concentrator as in claim 1, wherein the extruded structural component encloses a compressor.

5. An oxygen concentrator as in claim 1, wherein the extruded structural component includes integral mounting features for an electronic circuit board.

6. An oxygen concentrator as in claim 1, wherein the extruded structural component includes integral mounting features for a removable battery pack.

7. An oxygen concentrator as in claim 1, wherein the extruded structural component includes a chamber for product gas.

8. An oxygen concentrator as in claim 1, wherein the extruded structural component includes a chamber for purge gas.

9. An oxygen concentrator as in claim 8, wherein the chamber acts as a muffler.
